(19) United States
(12) Patent Application Publication
(10) Pub. No.: US 2021/0128061 A1
(43) Pub. Date: May 6, 2021

(54) METHODS AND DEVICES FOR CALCULATING HEALTH INDEX

(71) Applicant: BOMI LLC, Reno, NV (US)

(72) Inventor: Bomi JOSEPH, Los Gatos, CA (US)

(21) Appl. No.: 16/640,904

(22) PCT Filed: Aug. 21, 2018
(86) PCT No.: PCT/US2018/047289

§ 371 Date: Feb. 21, 2020

Related U.S. Application Data
(60) Provisional application No. 62/548,199, filed on Aug. 21, 2017.

(51) Int. Cl. A61B 5/00; A61B 5/0537; A61B 5/11; A61B 5/0205

(52) U.S. Cl. CPC A61B 5/4866; A61B 5/0537; A61B 5/0022; A61B 5/6807; A61B 5/112; A61B 5/021; A61B 5/4872; A61B 5/7475; A61B 5/742; A61B 2562/0285; A61B 5/0205

(57) ABSTRACT

Methods, systems, and devices for calculating a subject's health index and determining the subject's health status based on their health index. The disclosed systems include one or more wearable sensor units, each sensor unit comprising a pressure transducer component and an electrical impedance measuring component. The systems also include a network interface unit coupled to the one or more sensor units for transmitting data from the one or more sensor units, and a health index computing device coupled to the one or more sensors units for receiving data from the one or more sensor units. The health index computing device comprises a memory coupled to a processor which is configured to determine one or more physiological parameters based on data from the one or more sensor units, and measure the health index of the subject based on the determined weight and body fat composition of the subject.

This application claims the benefit of U.S. Provisional Patent Application Ser. No. 62/548,199, filed on Aug. 21, 2017, which is hereby incorporated by reference in its entirety.

### FIELD OF THE INVENTION

The present disclosure relates to methods, systems, and devices for calculating a subject's health index and determining the subject's health status based on their health index.

### BACKGROUND OF THE INVENTION

The simplest characteristics that can be measured for an individual are age, weight and height. Based on these, the Belgian Adolphe Quetlet proposed the concept of the Body Mass Index (BMI). He used it for comparing populations and stated that it was inadequate for use for individual diagnosis. Despite this, BMI has become very popular in evaluating the health of individuals.

BMI is defined as W/H², where W=Weight and H=Height, and it is a poor predictor of an individual's health. A study published Flegal et al, “Excess Deaths Associated with Underweight, Overweight, and Obesity,” JAMA 293 (15): 1861-7 (2005) showed that “overweight” (BMI=25-29.9) people had a similar relative risk of mortality to “healthy weight” (BMI=18-24.9) people as defined by BMI, while “underweight” (BMI<18) and “obese” (BMI>30) people had a higher death rate.

In an analysis of 40 studies involving 250,000 people, patients with coronary artery disease with “healthy weight” BMIs were at higher risk of death from cardiovascular disease than people whose BMIs put them in the “overweight” range (Romero-Coral Et al, August 2006). In the “overweight” range of BMI, the study found that BMI failed to discriminate between body fat % and lean muscle mass. The study concluded that the accuracy of BMI in diagnosing obesity is limited, particularly for individuals in the intermediate BMI ranges, in men and the elderly.

A review published by Flegal et al., “Association of All-cause Mortality with Overweight and Obesity using Standard Body Mass Index Categories: A Systemic Review and Meta-analysis,” JAMA 309(1): 71-82 (2013) has been widely quoted in the press claiming that “overweight BMI” people were healthier than “healthy BMI” people. Flegal reviewed 142 published articles with a combined sample size greater than 2.88 million and 270,000 deaths. This was a retrospective review and the various published studies had completely different experimental designs. It is impossible to distinguish the effects of the various factors and so it is not all clear how the confounding effect of the data has been resolved.

BMI has two significant deficiencies. It overestimates obesity in people with more lean body mass (false positives) and underestimates obesity on those with less lean body mass (false negatives). BMI is a very poor indicator of muscle mass and health. Patients with acceptable BMI have been found to have acceptable body weight, yet misleadingly high body fat percent and total cholesterol levels. Patients who have been classified as “overweight” and “obese” by BMI have been found to have greater than average lean body muscle mass, to have acceptable total cholesterol counts, and to be extremely muscular and fit. Accordingly, a new means for measuring human health is needed.

The present invention is directed as solving this and other deficiencies in the art.

### SUMMARY OF THE INVENTION

A first aspect of the present disclosure is directed to a system for monitoring a subject's health index. This system comprises one or more wearable sensor units, each sensor unit comprising a pressure transducer component and an electrical impedance measuring component; an interface unit coupled to said one or more sensor units for transmitting data from the one or more sensor units; and a health index computing device coupled to the one or more sensors units for receiving data from the one or more sensor units. The health index computing device comprises a memory coupled to a processor which is configured to be capable of executing programmed instructions comprising and stored in the memory to: determine one or more physiological parameters, based on said received data from the one or more sensor units, wherein said one or more physiological parameters include at least the subject's weight and the subject's body fat composition, and measure the health index of the subject based on the determined weight and body fat composition of the subject, and the subject's rest of body mass.

A second aspect of the present disclosure is directed to a method of measuring the health index of a subject. This method involves receiving, by a health index computing device, data from one or more sensors worn by said subject, said one or more sensors comprising a pressure transducer unit and an electrical impedance measuring unit. The method further involves determining, by the health index computing device, one or more physiological parameters related to the subject's health index based on the received data, wherein said physiological parameters include at least the subject's weight and body fat composition; and measuring, by the health index computing device, the health index of the subject based on the determined weight and body fat of the subject, and the subject's rest of body mass.

Another aspect of the present disclosure is directed to a non-transitory computer readable medium having stored thereon instructions for calculating a subject's health index comprising executable code which when executed by a processor, causes the processor to perform steps comprising receiving data from one or more sensor units worn by the subject. The one or more sensor units worn by the subject each comprise a pressure transducer unit and an electrical impedance measuring unit. One or more physiological parameters related to the subject's health index are determined based on the received data, where the physiological parameters include at least the subject's weight and body fat composition. The health index of the subject is measured based on the determined weight and body fat of the subject, and the subject's rest of body mass.

Another aspect of the present disclosure is directed to a health index computing device. The health index computing device comprises a processor and a memory coupled to the processor which is configured to be capable of executing programmed instructions comprising and stored in the memory to receive data from one or more sensor units worn by a subject, where the one or more sensor units each comprise a pressure transducer unit and an electrical impedance measuring unit. One or more physiological parameters related to the subject's health index is determined based on the received data, where the physiological parameters include at least the subject's weight and body fat composition. The health index of the subject is measured based on the determined weight and body fat of the subject, and the subject's rest of body mass.

### BRIEF DESCRIPTION OF THE DRAWINGS

FIG. 1 is an environment including a health index monitoring system of the present technology.

FIG. 2 is an exemplary block diagram of the one or more sensor units of FIG. 1.

FIG. 3 is an exemplary block diagram of the health index computing device as shown in FIG. 1.

FIG. 4 is an exemplary flow chart for a method of monitoring a subject's health index using the health index computing device.

FIG. 5 is a chart showing the correlation between health index and metabolic syndrome in men.

FIG. 6 is a chart showing the three zones for metabolic syndrome and health index in men.

FIG. 7 is a chart showing the three zones for metabolic syndrome and health index in women.

FIG. 8A is a graph showing the correlation between disease risk and health index in men. FIG. 8B is a graph showing the correlation between disease risk and health index in women.

FIG. 9 is a graph showing the number of men in a study population of 1466 having a BMI of less than 30 that were measured as “ideal” or “overweight”.

FIG. 10 is a graph showing health index vs. BMI in the study population of 1466 men.

FIG. 11A provides a graphical comparison of blood utilization during rest by individual males of the same height, but different health indices. FIG. 11B provides a graphical comparison of blood utilization during exercise by male individuals of the same height, but different health indices. FIG. 11C is a graph showing maximum blood flow (mL/kg/min) in male individuals of varying health indices. FIG. 11D is a table showing physiological data of the 21 male individuals, all 5′9″ but with varying health indices, that were studied to compare oxygen and blood flow, and caloric burn. FIG. 11E is a graph showing caloric burn at rest and during one hour of exercise by health index. FIG. 11F is a graph showing calories per pound of muscle per day that are burned at rest by male individuals of varying health indices. FIG. 11G is a graph showing the variation in calories per pound of muscle/hour that are burned during exercise in these same male individual of varying health indices.

FIG. 12A is a photograph of a left heel insert with a sensor unit as described herein containing pressure transducer components. FIG. 12B is a photograph of a full shoe insert with a sensor unit as described herein containing pressure transducer components.

FIG. 13 is a schematic of fabrication of a sensor comprising a pressure transducer component as described herein. The fabrication scheme and figure are adapted with modification from Gong et al., Nature Comm. 5:3132 (2014), which is hereby incorporated by reference in its entirety.

FIG. 14 is a circuit diagram of the impedance component of a sensor unit as described herein.

FIG. 15 is a flow diagram showing body fat measurement using the impedance component of a sensor unit as described herein.

FIG. 16 is a flow diagram showing detection of biomarkers in sweat using the pressure transducer component of the sensor unit as described herein.

### DETAILED DESCRIPTION OF THE INVENTION

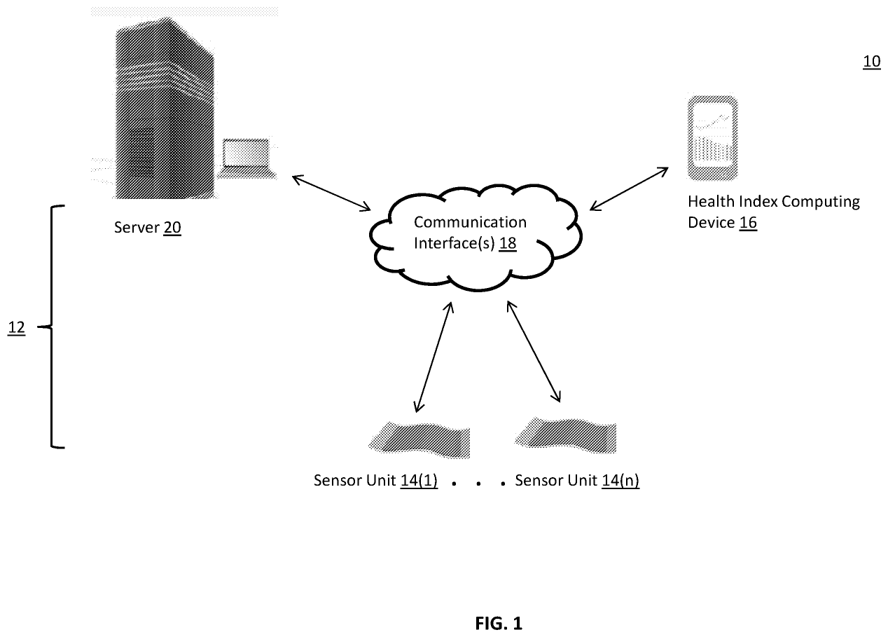

A first aspect of the present disclosure is directed to a system for monitoring a subject's health index. An exemplary environment 10 including an exemplary health index monitoring system 12 is illustrated in FIG. 1. This system includes, by way of example, one or more wearable sensor units 14(1)-14(n), each sensor unit comprising one or more sensor components capable of sensing or detecting one or more health index parameters of a subject wearing the sensor unit(s) 14(1)-14(n). The health index monitoring system 12 may include other types and/or numbers of devices, components, and /or elements in other combinations. This technology offers a number of advantages including methods and devices for providing real-time monitoring of a plurality of health index parameters and calculation of a health index score that is indicative of the subject's overall health status. The calculated health index score has prognostic and diagnostic value.

Referring more specifically to FIG. 1, this exemplary environment 10 includes the one or more wearable sensor units 14(1)-14(n) coupled to a communication interface unit 18 that transmits data from the one or more sensor units 14(1)-14(n) to a health index computing device 16 and/or another device, for example, a server 20 or storage device.

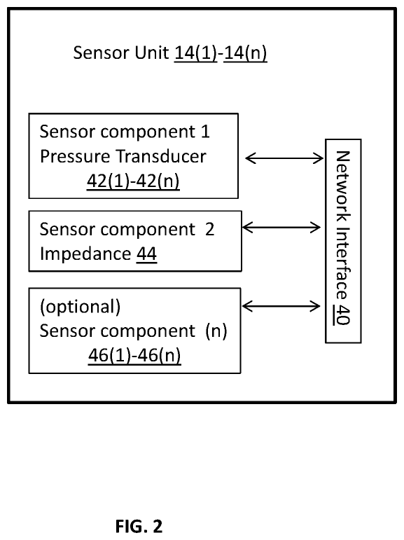

In reference to FIGS. 1 and 2 the one or more wearable sensor units 14(1)-14(n) of the system described herein each include one or more sensor components for detecting one or more health index parameters of a subject. In another embodiment, the one or more wearable sensor units 14(1)-14(n) each include two or more different sensor components for detecting two or more different health index parameters of the subject. In another embodiment, the one or more wearable sensor units 14(1)-14(n) each include at least three different sensor components for detecting at least three different health index parameters of the subject. For example, in one embodiment the wearable sensor unit 14(1)-14(n) includes one or more pressure transducer component(s) 42(1)-42(n) and an electrical impedance measuring component 44. In one embodiment the wearable sensor unit 14(1)-14(n) comprises one or more other biological sensors, such as one or more sweat analyzer components 46(1)-46(n), alone or in combination with the pressure transducer component(s) 42(1)-42(n) and/or the impedance measuring component 44.

The one or more pressure transducer component(s) 42(1)-42(n) of the one or more sensor units 14(1)-14(n) detect and resolve various forces including pressing, bending, torsion, and acoustic vibration at the macro- or microscopic level. The pressure transducer component(s) 42(1)-42(n) can be individually tuned to have various levels of sensitivity for the detection of various health index parameters including, but not limited to weight, blood pressure, heart beat, gait, and acoustical vibrations of the heart, lung, etc. A suitable pressure transducer component for sensing various pressing, bending, torsional, and acoustical vibration forces is composed of ultrathin gold nanowire coated graphene paper sandwiched between thin polydimethylsiloxane sheets as described herein. Such sensors can detect dynamic forces in a wide pressure range (10 Pa to 500 kPa), with a high sensitivity (>1/0.9 kPa microphone sensitivity), fast response times of less than 15 ms, and high stability over more than 50,000 repetitions, with low power consumption (i.e., less than 200 mW) when operated with a battery voltage of 1.5 V.

Alternative pressure sensor components suitable for incorporation into the wearable sensor unit 14(1)-14(n) described herein include those known in the art, for example, those described in Ghong et al., “A Wearable and Highly Sensitive Pressure Sensor with Ultrathin Gold Nanowires,” Nature Comm. 5:3132 (2014); Shuai et al., “Highly Sensitive Flexible Pressure Sensor Based on Silver Nanowires Embedded Polydimethylsiloxane Electrode with Microarray Structure,” ACS Appl. Mater Interfaces 9(31): 26314-26324 (2017); Joo et al., “Silver Nanowire-embedded PDMS with a Multiscale Structure for a Highly Sensitive and Robust Flexible Pressure Sensor,” Nanoscale 7(14): 6208-15 (2015); and Wang et al., “A Highly Sensitive and Flexible Pressure Sensor with Electrodes and Elastomeric Interlayer Containing Silver Nanowires,” Nanoscale 7(7): 2926-32 (2015), which are hereby incorporated by reference in their entirety.

The impedance measuring component 44 of the sensor unit 14(1)-14(n) of the system as described herein functions to measure the opposition to flow of an electric current through the body tissue of the subject wearing the sensor to obtain an estimation of total body water. This value is utilized to determine the percentage of body fat of the subject (see e.g., Kyle et al., “Bioelectrical Impedance Analysis—Part I: Review of Principles and Methods,” Clin. Nutrition 23(5): 1226-43(2004), which is hereby incorporated by reference in its entirety). In one embodiment, the impedance measuring component 44 comprises a circuit with three op amps, an instrumentation amplifier, and an impedance converter as described herein. In one embodiment, the bioelectrical impedance measurement is obtained from one wearable sensor unit comprising one electrode sending and receiving current flow through the body. In another embodiment, the bioelectrical impedance measurement is obtained using more than one wearable sensor where each sensor unit comprises an impedance electrode. The various sensors can function independently to obtain independent impedance values which are combined to obtain an average value. Alternatively, the various sensor can work in combination to measure electrical flow from a sensor unit located at a first position (e.g., the foot) to one or more sensors located at a second, third, or fourth location (e.g., foot, arm, trunk).

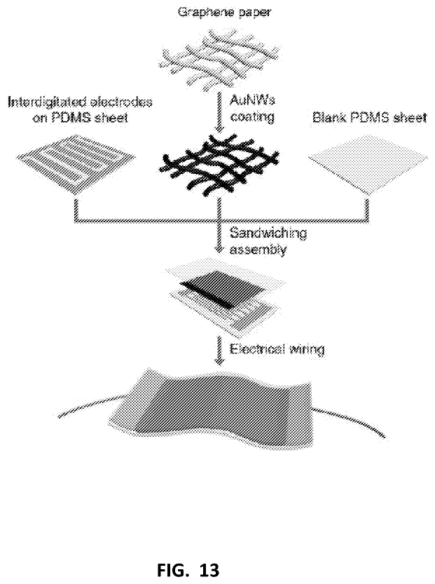

The sensor unit of the system described herein may further comprise a one or more sweat analyzer components 46(1)-46(n), each sweat analyzer component capable of detecting one or more biomarkers of health in eccrine sweat, for example, sweat rate, ions, small molecules, proteins and small peptides nucleic acid molecules, lipids, viral DNA, bacterial DNA and pathogens, hormones, and antibodies as described in more detail herein. The sweat analyzer component 46(1)-46(n) of the sensor unit may comprise graphene paper thinly coated with ultrathin gold nanowires and sandwiched between thin polydimethylsiloxane sheets as described herein (see, e.g., FIG. 13), where the structure of each sweat analyzer component is tuned to optimize sensitivity for the particular biomarker being detected. Alternative sweat analyzer units known in the art are also suitable for inclusion on the sensor unit described herein, see for example and without limitation, the sweat sensor unit described in Sonner et al., “Integrated Sudomotor Axon Reflex Sweat Stimulation for Continuous Sweat Analyte Analysis with Individuals at Rest,” Lab Chip 17:2550-2560 (2017); Gao et al., “Fully Integrated Wearable Sensor Arrays for Multiplexed in situ Perspiration Analysis,” Nature 529(7587): 509-514 (2016); Salvo et al., “A Wearable Sensor for Measuring Sweat Rate,” IEEE Sensors Journal 10(10): 1557 (2010); which are hereby incorporated by reference in their entirety.

In reference to FIG. 1 and FIG. 2, a network interface unit 40 is operatively coupled to the one or more sensor units for transmitting data from the one or more sensor units. In one embodiment, the network interface unit 40 operatively couples and facilitates communication between the one or more sensor units 14(1)-14(n) and the health index computing device 16. In another embodiment, the network interface unit 40 operatively couples and facilitates communication between the one or more sensor units 14(1)-14(n) and a storage device or a server 20. Other types and/or numbers of communication networks or systems with other types and/or numbers of connections and configurations can be used.

In one embodiment, the network interface unit 40 is a wireless interface unit. Suitable wireless communication technologies that can be included on the one or more sensor units of the system as described herein include, without limitation, Bluetooth, Zigbee, Zwave, 6 LowPan, Thread, Wifi, NFC, and cellular. For example, in one embodiment, the network interface unit 40 of the sensor unit is a Bluetooth device. In another embodiment, the network interface unit 40 is configured to communicate via WiFi.

In another embodiment, the interface unit is a non-wireless unit, such as USB unit. Other interface units known in the art for facilitating communication can also be utilized.

In another embodiment, the network interface unit 40 is a component of a microcontroller unit having processing capabilities that is coupled to the one or more sensor units. The microcontroller may execute a program of stored instructions for one or more aspects of the present technology as described herein, although other types and/or numbers of processing devices could be used and the microcontroller can execute other types and/or numbers of programmed instructions. The microcontroller unit of the sensor may also include electronics, such as A to D converters that allow the components of the sensor unit to communicate with the network interface unit.

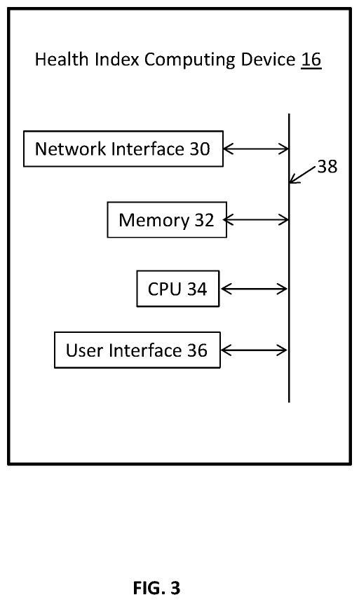

Referring now to FIG. 3, the health index computing device 16 of the system described herein can be any computing device, e.g., a computer, a personal computing device, smartphone, PDA, etc. that includes a central processing unit (CPU) or processor 34, a memory 32, and a network interface 30, which are coupled together by a bus 38 or other link. The health index computing device 16 may include other types and/or numbers of components and elements in other configurations.

The processor 34 in the health index computing device 16 executes a program of stored instructions for one or more aspects of the present technology as described and illustrated by way of the examples herein, although other types and/or numbers of processing devices could be used and the processor 34 can execute other types and/or numbers of programmed instructions. In one embodiment, the processor is located solely on the health index computing device. In another embodiment, the processor is distributed between the one or more sensor units and the health index computing device. For example, in one embodiment, the processor of the health index computing device comprises a microcontroller that is coupled to the one or more sensors. In this embodiment, the microcontroller serves as an on-board processor that is capable of mapping or converting data collected by the sensor into a digital signal that is transmitted to the health index computing device. The microcontroller coupled to the one or more sensors is capable of carrying out one or more processing functions of the health index computing device.

The memory 32 in the health index computing device 16 stores these programmed instructions for one or more aspects of the present technology as described and illustrated herein. A variety of different types of memory storage devices, such as a random access memory (RAM) and/or read only memory (ROM) in the timing processor device or a floppy disk, hard disk, CD ROM, DVD ROM, or other computer readable medium which is read from and written to by a magnetic, optical, or other reading and writing system that is coupled to the processor 34 in the health index computing device 16, can be used for the memory 32.

The network interface 30 of the health index computing device 16 operatively couples and facilitates communication between the health index computing device 16 and the one or more sensors 14(1)-14(n), although other types and/or numbers of communication networks or systems with other types and/or numbers of connections and configurations can be used. In one embodiment, the network interface unit 30 is a wireless interface unit. Suitable wireless communication technologies that can be included on the one or more sensor units of the system as described supra.

The health index computing device may further comprise a user interface 36, such as, for example, a graphical user interface, a touch user interface, or a web-based user interface. The use interface is configured to display information regarding the user's health index to the user. The user interface is also configured to receive input from the user regarding the user's health index. Information input by the user includes, but is not limited to information regarding sex, height, etc.

The server device 20 of the environment depicted in FIG. 1 and described herein can be one or a plurality of computing devices that each include a CPU or processor, a memory, and a network interface, which are coupled together by a bus or other link. The server device may include other types and/or numbers of components and elements in other configurations.

The processor of the one or more server devices 20 executes a program of stored instructions for one or more aspects of the present technology as described and illustrated by way of the examples herein, although other types and/or numbers of processing devices could be used and the processor can execute other types and/or numbers of programmed instructions.

The memory of the one or more server devices 20 stores these programmed instructions for one or more aspects of the present technology as described and illustrated herein. A variety of different types of memory storage devices, such as a random access memory (RAM) and/or read only memory (ROM) in the timing processor device or a floppy disk, hard disk, CD ROM, DVD ROM, or other computer readable medium which is read from and written to by a magnetic, optical, or other reading and writing system that is coupled to the processor(s) of the one or more server devices 20, can be used for the memory.

The network interface of the one or more server devices 20 operatively couples and facilitates communication between the server device(s) 20 and the one or health index computing devices 16 and/or the one or more sensor units 14(1)-14(n), although other types and/or numbers of communication networks or systems with other types and/or numbers of connections and configurations can be used. In one embodiment, the network interface unit is a wireless interface unit. Suitable wireless communication technologies that can be included on the one or more sensor units of the system as described supra.

Communication network 18 of the system described herein can include one or more local area networks (LANs) and/or wide area networks (WANs). By way of example only, the communication network 18 can use TCP/IP over Ethernet and industry standard protocols, including hypertext transfer protocol (HTTP) and/or secure HTTP (HTTPS), although other types and/or numbers of communication networks may be utilized.

In addition, two or more computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on computer system(s) and/or cloud computing devices that extend across any suitable network using any suitable interface mechanisms and traffic technologies, including by way of example only teletraffic in any suitable form (e.g., voice and modem), wireless traffic networks, cellular traffic networks, Packet Data Networks (PDNs), the Internet, intranets, and combinations thereof

Aspects of this technology may also be embodied as a non-transitory computer readable medium having instructions stored thereon as described and illustrated by way of the examples herein, which when executed by a processor, cause the processor to carry out the steps necessary to implement the methods of the examples, as described and illustrated herein.

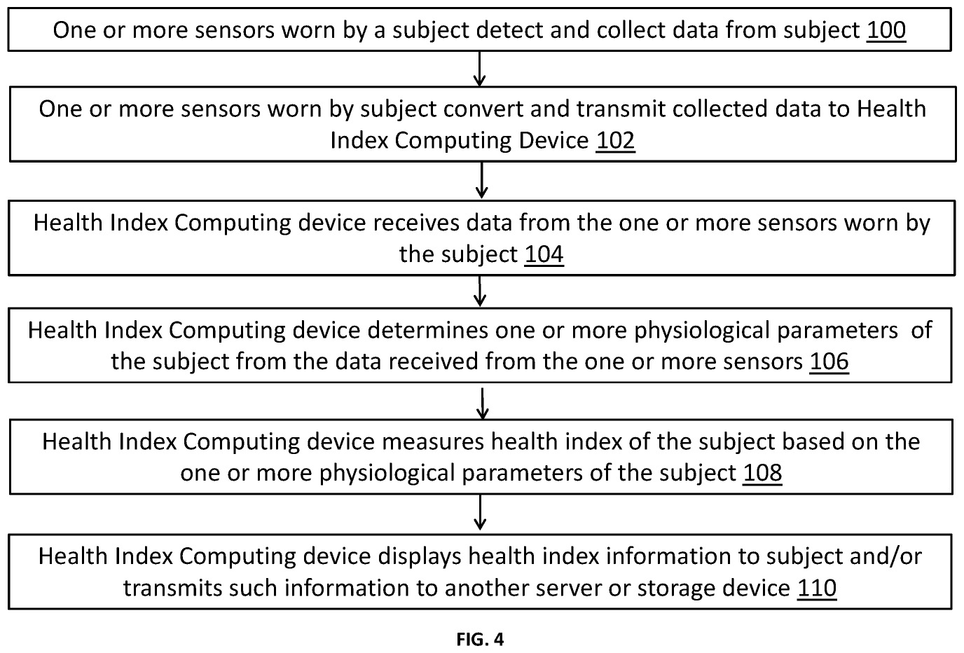

An exemplary method of determining and monitoring a subject's health index will now be described with reference to FIGS. 1-4. In particular FIG. 4 is a flowchart showing an exemplary method for monitoring a subject's health index. In accordance with this method, the one or more wearable sensor units 14(1)-14(n) worn by a subject detect and/or collect health index related data from the subject 100. Exemplary sensor units for detecting and collecting health index related data from the subject are described in more detail herein. Briefly, each sensor unit comprises one or more sensor components capable of detecting and collecting different physiological data from the individual wearing the sensor. In one embodiment, each sensor unit comprises two or more sensor components, each component capable of detecting and collecting different physiological data. In another embodiment, each sensor unit comprises at least three different sensor components, each component capable of detecting and collecting different physiological data. The sensor components of the sensor unit are coupled to a network interface that facilitates transfer of the collected data to one or more other devices, including a health index computing device.

The data that is collected by the one or more sensor units 14(1)-14(n) is converted to a digital signal and transmitted to the health index computing device 16 via the communications interface 18 in step 102, and the health index computing device 16 receives the data from the one or more sensors 14(1)-14(n) in step 104. In one embodiment, the conversion of analog data to a digital signal occurs within a microcontroller unit containing an A/D converter unit of the one or more sensor units.

In the next step 106, the health index computing device 16 determines one or more physiological parameters of the subject from the data received from the one or more sensors. For example, in one embodiment, the health index computing device is capable of processing and converting the digital signal received from the pressure transducer component of a sensor unit into a weight value for the subject wearing the sensor. In another embodiment, the health index computing device is capable of processing and converting the digital signal received from the impedance sensor component of the sensor unit into a body fat composition value for the subject wearing the senor. As described supra, the health index computing device can be any computing device, including, without limitation, a computer, personal computing device, smartphone, PDA, etc., containing a memory, CPU, and network interface.

In the next step, the health index computing device 16 measures the health index of the subject as described herein based on the one or more physiological parameters of the subject 108. The formula and means for measuring the health index of the subject is described in more detail herein. Finally, the health index computing device 16 displays health index information to the subject and/or transmits such information to a server 20, such as a third party server.

In reference to FIG. 4, in an exemplary method of the present disclosure, the one or more wearable sensor units 14(1)-14(n) include at least one or more pressure transducer component(s) 42(1)-42(n) and an impedance measuring component 44. In accordance with this embodiment, in Step 100, the one or more pressure transducer components 42(1)-42(n) and impedance measuring component 44 detect one or more signals from the subject, e.g., a pressure signal and an impedance signal. These signals are converted from an analog to a digital form and transmitted directly or via a microcontroller to the health index computing device (Step 102). The health index computing device 16 receives the data from the one or more sensors 14(1)-14(n) for processing (Step 104). In accordance with this example, in Step 106, the health index computing device 16 determines at least the subject's weight and subject's body fat composition from the digital data received from the one or more sensor units. Other physiological parameters that can also be detected by the sensor units include, without limitation, the subject's gait, blood pressure, pulse, heart beat, etc. as described herein.

In Step 108, the health index computing device 16 measures the health index of the subject based on the subject's weight, percent body fat, and other parameters, including the subject's rest of body value, as described in more detail below.

As used herein, a subject's “health index” refers to the ratio of muscle mass to body fat of the subject.

Health Index=Muscle Mass/Body Fat

More particularly, the health index of an individual is the neuromuscular tissue to visceral fat ratio of the subject. To accurately calculate this ratio, the subject's muscle mass must be determined. Muscle mass can be determined by subtracting the subject's body fat (detected by the impedance component of the one or more sensor units) and the subject's rest of body (ROB) tissue weight from the subject's body weight (as detected by the pressure transducer component of the one or more sensor units).

Muscle Mass=Body Weight−Body Fat−ROB

As used herein, “rest of body” (ROB) mass refers to the mass of bodily tissue and fluid other than muscle and fat, i.e., ROB mass comprises the mass of the bones, cartilage, tissue, blood, of an individual. The ROB is a relatively constant value. In one embodiment, the ROB mass for a man is between 0.5154-0.5201 of his ideal weight and the ROB mass for a woman is between 0.4686-0.4818 of her ideal body weight. The ideal body weight of an individual can be calculated using any standardized reference chart or formula known in the art, for example and without limitation, G. J. Hamwi's Formula from 1964, B. J. Devine's Formula from 1974, J. D. Robinson's Formula from 1983, and D. R. Miller's Formula from 1983.

In one embodiment, the processing unit (CPU) of the health index computing device computes the health index using the body weight and body fat data received from the one or more sensors in combination with ROB and data input by the user using the health index formula above (Step 108). The health index information is displayed via the graphical user interface of the health index computing device to the user (Step 110). The health index computing device stores and tracks the health index data from the subject in real time. In addition, the network interface of the health index computing device transmits the health index information of the user to a cloud, server, or other storage device (Step 110).

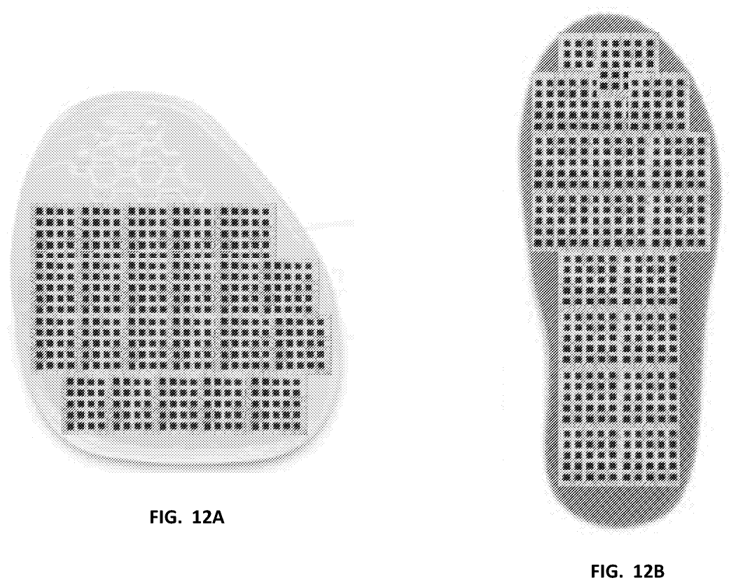

The one or more sensor units as describe herein detect and collect a variety of physiological data of the subject wearing the sensor units. In one embodiment, the sensor unit, comprising the pressure transducer component as described herein, measures weight and weight displacement. Weight and weight displacement can be measured using single sensor unit comprising a pressure transducer component placed in contact with the ventral surface of the subject's foot, e.g., the heel of the subject (see e.g., heel insert as shown in FIG. 12A). In another embodiment, weight and/or weight displacement are measured using two sensor units, each sensor unit comprising a pressure transducer component, placed in contact with both heals of the individual. The weight readings of the two sensors are averaged for a more accurate reading.

The one or more sensor units comprising one or more pressure transducer components as described herein can be utilized to detect bending and torsional forces of the individual. In one embodiment, these forces are measured using a sensor unit taking the form of a whole shoe insert. Quantitative gait analysis is a very useful tool for athletes and clinicians to monitor and evaluate patient's recovery. The gait characteristic of individuals can also be monitored in an individual as they are changing their health index. An athlete can use gait measurement to improve their form and performance. Doctors can use gait analysis to rehabilitate patients with training devices or prosthetics.

The sensor units comprising the pressure transducer component described herein can measure tri-axial bending and torsion forces to assess real time gait characteristics of the individual over the complete ground contact point of the feet. This offers an advantage over currently available devices which typically contain two to eight analog pressure transducer sensors. Every individual's feet differ and the placement of the sensors become problematic. Accordingly, in this embodiment, a full foot pad (FIG. 12B) that serves as a 360° potentiometer (measuring angles of bend) acts as a continuous signal generator (akin to an analog device rather than a discrete digital device) and a real-time pressure sensor.

The one or more sensor units comprising a pressure transducer component as described herein can also be utilized to detect pulse and heart rate of the individual. The subtle differences in blood pulses that can be identified indicate the device can be utilized to detect, diagnose, and monitor, in real-time, a variety of cardiovascular diseases, such as angina, myocardial infarction, stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis.

The one or more sensor units comprising a pressure transducer component as described herein can also be utilized to measure acoustic vibrations of the lungs, pulse, and heart. Detection of arterial pulse, heart rate etc are rate dependent measurements. A heart rate does not give a doctor the acoustic characteristic of the heart beat. In other words, it does not simulate the acoustic feel of the heart that the doctors get when they listen through a stethoscope. The pressure transducer component of the sensor unit described herein serves as a real-time phonocardiogram (PCG) which accurately replicates electrocardiograph (ECG) studies of the heart (Debbal S. M., “Model of Differentiation between Normal and Abnormal Heart Sounds in Using the Discrete Wavelet Transform,” Journal Medical and Bioengineering 3(1): 3-11 (2014), which is hereby incorporated by reference in its entirety. Accordingly, the sensor unit containing the pressure transducer component as described herein can also be utilized to detect and monitor, in real-time, heart defects such as aortic stenosis, mitral stenosis, aortic regurgitation and mitral regurgitation, arterial incompetence, mitral incompetence. The sensor unit containing the pressure transducer component as described herein can also provide an early warning of stroke, heart failure, myocardial infarction, coronary artery disease, hypertension, cardiomyopathy, valve defect and arrhythmia (atrial fibrillation, atrial flutter, sick sinus syndrome, sinus tachycardia, atrial tachycardia, ventricular fibrillation, premature contractions, heart block, syncope).

There is a strong connection between the heart and the lung. The same phonocardiogram principal can also be applied to phonopneumography to detect bronchitis, COPD, asthma etc.

Non invasive and accurate access of biomarkers remains a holy grail of the medical community. Human eccrine sweat is a biomarker-rich fluid which is gaining increasing attention. The sensor unit described herein containing the sweat analyzer component is capable of continuous bio-monitoring which is not possible with other biofluids such as invasive blood monitoring.

Sweat as a biomarker has been used to detect cystic fibrosis (Sato et al., “Biology of Sweat Glands and their Disorders. I. Normal Sweat and Gland Function,” J Am. Acad. Dermatol. 20: 537-563 (1989), which is hereby incorporated by reference in its entirety) and metabolites of illicit drugs (De Giovanni and Fucci, “The Current Status of Sweat Testing for Drugs of Abuse: A Review,” Curr. Med. Chem. 20:545-561 (2013), which is hereby incorporated by reference in its entirety). The main limitations of sweat as clinical sample are the difficulty to produce enough sweat for analysis, sample evaporation, lack of appropriate sampling devices, need for a trained staff, and normalization of the sampled volume. Many of these issues are solved by the sweat analyzer component of the sensor unit as described herein, where the gold nanowires are in immediate contact with sweat as it emerges onto skin. This not only improves convenience and eliminates evaporative issues, which cause misleading increases in biomarker concentrations, but is also powerful in collecting many biomarkers which degrade in as little as 10-20 min. The sweat analyzer component described herein can also easily measure the change in sweat generation rate, tracks sweat generation rate, actual biomarker sampling, and keeps track of detailed microfluidic and transport model between the sweat glands and sensors. This is critical because it allows the discovery of new biomarkers in sweat.

All historical studies have used classic crude clinical techniques such as collection of large sweat volumes in bags or textiles. As a result, many biomarkers are discarded prematurely due to an apparent lack of correlation between blood and sweat. The transport rate of a biomarker into sweat is passive (diffusive) and slower than the rate at which the biomarker is transported to the skin surface. Lactate, for example, is of great interest in both athletics and critical care applications. The metabolic activity of a hardworking sweat gland itself creates abundant lactate which dominates over lactate diffusion from plasma. High exertion exercise tests can therefore show a spike in lactate correlating with exercise intensity (sweat rate), but this does not necessarily represent the anaerobic state of the body.

There is also a major problem of the contamination of the markers from the skin surface. It has been shown that glucose levels in sweat do trend well with glucose levels in blood. However, a severe limitation has been that glucose diffusing from the uppermost layers of skin into the wet (sweaty) skin surface completely confounds the sweat glucose correlation with blood. The sweat analyzer component of the sensor unit described herein solves this problem by isolating the analyzed sweat from the skin surface contaminants. The sweat analyzer as described herein can continuously, non intrusively and accurately, monitor the glucose levels of a diabetic (any human) by monitoring their sweat, without any necessity of sampling their blood.

In one embodiment, the sweat analyzer component of the sensor unit described here detects and measures sodium chloride ions in sweat in real-time. Sodium and chloride ions are the most abundant solutes in sweat. Sweating is an active process which is fundamental to the secretion of water. Water and NaCl are partitioned from blood into the lumen of the secretory coil through several steps and is finally pumped along the length of the sweat duct. The body has developed mechanisms to retain electrolytes, which would normally be rapidly lost when large volumes of sweat are used for cooling the body. Most sports drinks are marketed under the flawed notion that our body does not recycle sodium and chloride. Sodium and chloride ions are actively reabsorbed via the epithelial sodium channel and the CF transmembrane regulator. Levels of both sodium and chloride are elevated at high sweat rates, indicating that the two channels that reabsorb sodium and chloride ions work at-or-near the same rates regardless of the sweat flow rate in the duct.

Measuring sodium and chloride in sweat provides correlations with concentrations or conditions in blood. Chloride levels in sweat can be used to predict hormonal changes and potentially ovulation. If sodium or chloride are not reabsorbed properly, havoc reigns. In cystic fibrosis, the cystic fibrosis transmembrane regulator channel does not function properly. Chloride does not get reabsorbed sufficiently and is evident from the increased sweat chloride concentrations. Cystic fibrosis is easily, non-invasively, detected by testing the sweat for higher chloride concentration.

Another useful reason to measure sodium and chloride in sweat is because the concentrations can be used as a direct measure of sweat rate. Measuring sweat rate is important for improved measures of biomarkers whose concentrations are sweat rate dependent. It is important for measuring the effective sampling interval for biomarkers. It is also a real indicator of the water and salt loss for an athlete or any person in inclement weather. It will warn against hyperthermia well in advance and is quite accurate.

In one embodiment, the sweat analyzer component of the sensor unit described here detects and measures the passive ion partitioning of potassium. Potassium is a small ion like sodium and chloride but its transport mechanism is vastly different. Potassium in plasma predicts muscle activity and the imbalance of potassium causes hypo- or hyperkalemia. Potassium concentration in sweat is proportional to blood concentration and was found to be independent on sweat rate. The precise method of potassium partitioning into sweat is not known. The concentration at the surface of skin is likely caused by potassium leaks into the duct and is a very accurate method of measuring physical activity.

In one embodiment, the sweat analyzer component of the sensor unit described here detects and measures passive ammonia partitioning. Ammonia correlates strongly with exercise intensity and blood levels. Ammonia is also substitute for lactate measurement of anaerobic condition. Ammonia (NH3) passes through the cells lining the secretory coil by means of a passive diffusion. The low pH of sweat causes most ammonia molecules to protonate to ammonium (NH4). While NH₃ has a high level of diffusivity through cellular membranes, NH₄ does not because of its electrical charge. This causes “trapping” of ammonium and prevents it from being absorbed back in the body. The concentration of ammonium (NH4) in sweat is 20-50 times higher than the concentration of ammonium in plasma. If sweat is not rapidly measured, collected and sealed, carbon-dioxide in the atmosphere is absorbed and makes it more acidic. This has caused errors in some scientific studies but does not affect real time measurements using the sweat sensor as described herein.

In one embodiment, the sweat analyzer component of the sensor unit described here detects and measures passive small molecules. Ethanol, the molecule which causes intoxication has been well studied. Using the sweat sensor component described herein, the results of several studies showing a strong correlation between blood and sweat ethanol concentrations were verified. This enables continuous blood alcohol (BAC) analysis by sweat measurement. Ethanol concentrations in sweat were monitored and a higher sensitivity compared to breathalyzers commercially available was demonstrated. The sweat analyzer component described herein showed a response to sweat ethanol concentrations within 4 minutes after ingestion of an alcoholic beverage.

The hydrophilic nature of ethanol causes ethanol to permeate through most membranes within the human body rapidly. Ethanol concentration were found to be about 20% more concentrated in sweat compared to blood. This is because sweat has approximately 20% more water than that of blood. When comparing the concentration of ethanol in terms of milli-moles per liter of water (instead of liter of solution), sweat and blood ethanol concentrations correlate one to one. This simple result reinforces the point that correlations with blood should always consider the fact that blood has a lesser percentage of water compared to sweat.

In one embodiment, the sweat analyzer component of the sensor unit described here detects and measures passive small molecules. Cortisol is a vital hormone, released by the hypo-thalamo-pituitary-adrenal complex, inbresponse to stress. cortisol's presence can be detected in many bodily fluids including sweat. Unbound cortisol is able to diffuse through membranes, while cortisol bound to carrier proteins is unable to make this diffusion. Our studies show that sweat cortisol level correlates with unbound serum cortisol levels. Cortisol levels in sweat range from 2.21*10⁻⁵ to 3.86*10⁻⁴ mM, with the greatest concentration being found in the morning. Comparative blood cortisol concentrations ranged from 1.24*10⁻⁴ to 4.0*10⁻⁴ mM. Type 2 11-β-hydroxysteroid-dehydrogenase (HSD), the enzyme capable of converting cortisol to cortisone, was also detected. Therefore, the sweat analyzer as described herein can also measure, quantify and display (in real time), the level of stress perceived by an individual.

In another embodiment, the sweat analyzer component of the sensor unit described here detects and measures the small passive molecule urea. Urea is the nitrogen-containing metabolite, typically excreted by the kidney. It is also found to be alternatively excreted through sweat glands. Urea from sweat is even visibly observable as a white skin crust in patients suffering from kidney failure. Their urea levels are up to 50 times greater than in serum. Urea concentrations, in sweat, for healthy males was in the range of 2 to 6 mM, and their serum urea concentrations ranged from 5 to 7mM. At low sweat rates, their sweat: plasma urea concentration reached an upper limit of 4 but it approached 1 as sweat rates increased.

Urea is one of the primary components of urine. It is an excess of amino acid and protein metabolites, such as urea and creatinine, in the blood that normally should have been excreted in the urine. Both uremia and the uremic syndrome have been used interchangeably to define renal failure.

In one embodiment, the sweat analyzer component of the sensor unit described here detects and measures small molecules, such as lactate, that are generated by a gland. Lactate is a small metabolite produced as a result of anaerobic activity. High exertion exercises and critically ill patients have high amounts of lactate. Lactate is used to produce energy in the absence of adequate oxygen. Sweat lactate is an indirect indicator of body exertion and may be directly related to exertion of the sweat gland in response to whole body exertion. Sweat lactate levels, after exercise, ranged from a low of 6 mM to a high of 100 mM, depending on the length and intensity of the anaerobic workout.

Sweat lactate levels can be used as an indicator of anaerobic workout load for healthy individuals. They can also be used to monitor the health of critically ill patients.

In another embodiment, the sweat analyzer component of the sensor unit described here detects and measures peptides and small protein partitioning. Many peptides and small proteins, which correlate with plasma, have been detected in the pure eccrine sweat. Larger protein molecules, with a 3 dimensional structure, are not seen in sweat. Cytokines are reduced regulatory proteins synthesized and released by immune system cells as well as a variety of other cells. Interleukin-6 (IL-6), IL-1α, IL-1β, IL-8, tumor necrosis factor-α (TNF-α), and transforming growth factor-β (TGF-β) have been detected in eccrine sweat and have been shown to have a direct correlation with plasma levels (Marques-Deak et al., “Measurement of cytokines in sweat patches and plasma in healthy women: Validation in a controlled study,” J. Immunol. Methods 315: 99-109 (2006), which is hereby incorporated by reference in its entirety).

Peptides such as neuropeptide Y (NPY) also display correlation between sweat and blood. NPY is a polypeptide found in dermal nerve fibers, sweat glands, and hair follicles. NPY is found to exhibit anti-stress activities when receptors are activated, along with numerous other effects such as anti-depressive properties. In a study of women with major depressive disorder (MDD) in remission, levels of cytokine and neuropeptide concentrations were measured via sweat patch and plasma comparisons (Cizza et al., “Elevated neuroimmune biomarkers in sweat patches and plasma of premenopausal women with major depressive disorder in remission: The POWER study,” Biol. Psychiatry 64: 907-911 (2008), which is hereby incorporated by reference in its entirety). It was found that sympathetic NPY increased from 4.45*10⁻¹⁰ mM, in sweat of healthy patients, to 1.19*10⁻⁸ mM in sweat of MDD patients. The plasma levels in both groups were comparable. This is a case in which sweat analysis was more indicative than plasma analysis.

Given the ability to sense neuropeptides, hormones, peptides, small proteins, inorganic salts, organic molecules, DNA, RNA, etc., the sweat sensor as described herein can be utilized to detect, diagnose, and monitor the diseases listed in Table 1.

TABLE 1
List of Diseases that can be detected, diagnosed and monitored
using the methods and devices described herein.

| | | |
|---|---|---|
| Asthma | Allergies | Parkinsons |
| Auto immune disease | Anxiety | PTSD |
| Bipolar disorder | Arthritis | Rheumatoid arthritis |
| Cardiovascular disease | Cholesterol | Schizophrenia |
| Diabetis | COPD | Trigeminal neuralgia |
| Epilepsy | Depression | Migraines |
| Glaucoma | Emesis | Osteoarthritis |
| Gout | Endometriosis | Osteoporosis |
| Kidney failure | Fibroids | Ovarian cysts |
| Liver Disease | High Blood Pressure | Schizophrenia |
| Menopause | Inflamation | Ulcerative colitis |
| Multiple Sclerosis | Insomnia | 162 different cancers |
| Muscular degeneration | Irritable bowel syndrome | |
| Neurodegeneration | Low bone density | |
| Neuroprotection | Low immunity | |

Another aspect of the present disclosure is directed to a biological sensor unit. The biological sensor is composed of a nanowire coated graphene core. In one embodiment, the biological sensor is composed of a gold nanowire coated graphene core. In another embodiment, the biological sensor is composed of silver nanowire coated graphene core. Alternative nanowires that are suitable for use in the biological sensor as disclosed herein include, without limitation, nickle nanowires (NiNW), platinum nanowires (PtNW), silicon nanowires (SiNW), Indium phosphide nanowires (InPNW), and gallium nitride nanowires (GaNNW). The thickness of the nanowire coating on the graphene core can range from 1 nM-1000 nM, which controls, in combination with other variables, the sensitivity of the biological sensor to detect diverse parameters at macroscale or microscale levels. Other variables that affect the sensitivity of the biological sensor include (i) the thickness of the nanowires, which range from 0.0126 mm (AWG 28 gauge) to 0.808 mm (AWG 12 gauge), the inter spatial distance between the edges of the wires (with the coating), which typically ranges from 0.1 mm to 0.5 mm, and (iii) the thread count (i.e., number of threads per inch), which ranges from 100 to 500 threads/inch. Modulating the aforementioned variables, the sensor can be tuned to detect parameters such as pressure to measure weight, shear, torque. Alternatively, it can be tuned to detect parameters such as electrolyte and/or proteins concentrations in a sample.

The nanowire coated graphene core of the sensor is sandwiched between polymeric sheets interdigitated with an electrode array. Suitable polymeric materials include, without limitation, polydimethylsiloxane (PDMS), poly(methyl methacrylate) (PMMA), polycarbonates (PC), epoxy-based resins, copolymers, polysulfones, elastomers, cyclic olefin copolymer (COC), and polymeric organosilicons.

Another aspect of the present disclosure is directed to a method of determining a subject's health index. This method involves calculating the subject's health index using the health index formula as described supra and shown below:

Health Index=Muscle Mass/Body Fat

The subject's muscle mass can be measured directly using a method such as total body potassium count (Wang et al., “A New Total Body Potassium Method to Estimate Total Body Muscle Mass in Children,” J. Nutr. 137(8):1988-1991 (which is hereby incorporated by reference in its entirety), MRI (Ross et al., “Influence of Diet and Exercise on Skeletal Muscle and Visceral Adipose Tissue in Men,” J. Appl. Physiol. 81: 2445-55 (1996), which is hereby incorporated by reference in its entirety), or computed tomography (Bulcke et al., “Computed Tomography of the Human Skeletal Muscular System,” Neuroradiology 17:127-136 (1976), which is hereby incorporated by reference in its entirety). Alternatively, the subject's muscle mass can be determined using the formula described supra and shown below:

Muscle Mass=Body Weight−Body Fat−ROB

The subject's body weight and body fat composition can be determined using the sensor device as described herein or alternative methods known in the art. For example, and without limitation, the subject's body fat can be accurately measure using skin calipers, hydrostatic weighing, bioelectrical impedance, dual-energy X-ray absorptiometry (DEXA), or air-displacement plethysmography (ADP). As described supra, ROB (rest of body mass) refers to the mass of bodily tissue and fluid other than muscle and fat. The ROB is a relatively constant value. In one embodiment, the ROB mass for a man is between 0.5154-0.5201of his ideal weight and the ROB mass for a woman is between 0.4686-0.4818 of her ideal body weight.

A subject's health index, calculated using the systems and methods described herein, serves as an indicator of the subject's overall health status. As described in the examples herein, a comparison of health index to body mass index (BMI) showed that the health index is a far more discerning measure of health than BMI. The health index picks up the transition from health to unhealthy that the BMI does not. Accordingly, another aspect of the present disclosure is directed to a method of determining a subject's health status. This method involves determining the subject's health index as described herein. For a male subject, a health index value of 0-2.3 indicates poor health status, a health index value of 2.3-4 indicates a transitional health status, and a health index value of above 4 indicates an good health status. For a female subject, a health index value of 0-1.3 indicates poor health status, a health index value of 1.4-2 indicates a transitional health status, and a health index value of above 2 indicates a good health status.

Another aspect of the present disclosure is directed to a method of detecting, diagnosing, and/or prognosing disease in a subject. This method involves determining the health index of the subject using the methods described herein and detecting, diagnosing, and/or prognosing disease in the subject based on the determined health index value.

As described in the examples herein, the health index has been correlated to factors contributing to metabolic syndrome and disease. Metabolic syndrome is widely acknowledged by all medical organizations as a major indicator of disease. It is a cluster of conditions—increased blood pressure, high blood sugar, excess body fat around the waist, and abnormal cholesterol or triglyceride levels that when occurring together, significantly increase your risk of heart disease, stroke and diabetes. Having just one of these conditions is not metabolic syndrome. Having more than one of these increases your risk even more. You are considered positive for metabolic syndrome if you have positive for three or more conditions.

For example, for a male subject, a health index value of 0-2.3 indicates the presence of >2 metabolic conditions contributing to metabolic syndrome. A health index value of 2.3-4 indicates the presence of 0-2 metabolic conditions contributing to metabolic syndrome, and a health index value of above 4 indicates the presence of no metabolic conditions (i.e., no metabolic syndrome). For a female subject, a health index value of 0-1.3 indicates the presence of >2 metabolic conditions contributing to metabolic syndrome. A health index value of 1.4-2 indicates the presence of 1-2 metabolic conditions contributing to metabolic syndrome, and a health index value of above 2 indicates the presence of no metabolic conditions (i.e., no metabolic syndrome).

### EXAMPLES

Examples are provided below to illustrate the present invention. These examples are not meant to constrain the present invention to any particular application or theory of operation

### Example 1

### Calculation of a Subject's Health Index

In this study the health index as defined herein (Health Index=(Muscle Mass)/(Body Fat) was analyzed in 1466 men and 2678 women. All the men and women had a BMI in the “healthy” or “overweight” range. This was done to analyze the conflicting reports of people in this BMI range. Subjects in the “obese” range were not chosen because there is very little controversy about the negative health implications of being “obese”.

Body Fat Measurement by Air Displacement Plethysmography (ADP): The BODPOD from Life Measurement Inc., Concord, Calif., USA, with software version 2.23 was used with the manufacturer's recommendations and guidelines for calibration and measurement. Each subject had their % fat mass (FM) and fat free mass (FFM) evaluated by ADP. Each subject wore a swimsuit and cap provided by us and their body mass was measured to the nearest 0.1kg by an electrical scale connected to the ADP computer.

The thoracic gas volume was calculated as—

Thoracic Gas Volume=Functional Residual Capacity+0.5*Tidal Volume Body Density (BD) was calculated as Body Mass/Body Volume

Fat % by ADP was calculated using Siri's (1961) equation—

ADP % Fat=[(4.95/BD)−4.50]* 100

Total Muscle Mass Measurement by Total Body Potassium (⁴⁰K) Count: The whole body ⁴⁰K counting method outlined by Wang et al., “Whole Body Skeletal Muscle Mass: Development and Validation of Total-Body Potassium Prediction Models,” Amer. J. Clin. Nutri. 77(1):76-82 (2003), which is hereby incorporated by reference in its entirety, was utilized to measure total muscle mass. Hansen et al., “Determination of Skeletal Muscle and Fat Free Mass by Nuclear and Dual X-ray Absorptiometry Methods in Men and Women Aged 51-84,” Amer. J. Clin. Nutri. 70(2): 228-33 (1999) showed no significant differences in Total Muscle Mass (TMM) measured by DEXA, ⁴⁰K and Potassium Nitrogen (KN) methods. However, the ⁴⁰K measurements pick up short term changes to protein levels better than DEXA and KN methods.

Subjects included in this study had no physical ailments or orthopedic handicaps. Each subject completed a medical history, had a physical examination and had a comprehensive metabolic profile (CMP) blood test. Body mass was measured to the nearest 0.1 kg. All weight and CMP tests were done while on a 12 hour fast. Height was measured by a Stadiometer to 0.1 cm accuracy.

The whole body counter was used to measure the natural 1.46 MeV ⁴⁰K γgrays. The ⁴⁰K raw counts were collected over 9 minutes and adjusted for body size on the basis of the calibration equation presented by Pierson et al., “Body Potassium by Four-pi 40K Counting: An Anthropometric Correlation,” Am. J. Physiol. 246(2 Pt 2):F234-9 (1984), which is hereby incorporated by reference in its entirety.

The corrected equation used for men was as follows:

Corrected Body K (men) or TBK=0.85*⁴⁰K+7.6*weight+250

The corrected equation for women used was—

Corrected Body K (women) or TBK=0.88*⁴⁰K+6.4*weight+64.2

The assumptions made for calculating total muscle mass (TMM) were—

a) Total body potassium (TBK) is measurable as ⁴⁰K

b) A stable and known TBK to TMM ratio exists in healthy adults

c) Observed TBK mean values were 120.1 mmol/kg for men and 119.4 mmol/kg for women (r=0.98, P<0.001)

Total muscle mass (TMM) was calculated as—

TMM=0.0082*TBK (Wang et al, 2003)

Rest of Body Mass Calculation: A subject's muscle mass was also measured using the following formula:

Muscle Mass=Body Weight−Body Fat−ROB

As described herein, ROB (rest of body) mass is the mass of body tissue and fluid other than muscle and fat. The mean sample ROB mass for adult men was found to be 0.5178 of their ideal weight W₁. The sample size was 1466 and included races diverse as Caucasians, African-Americans, Asians, Polynesians, Native

Americans and South Asian Indians. The variance (s²) was 0.0021 and the standard deviation (s) was a narrow 0.04554. The coefficient of variance was only 8.79%. The median was 0.5175 and the midrange was 0.5335. With 95% confidence it can be predicted that the mean population ROB mass for men exists between the values 0.5154 and 0.5201.

The mean sample ROB mass for adult women was found to be 0.4762 of their ideal weight W₁. The sample size was 2678 and included races diverse as Caucasians, African-Americans, Asians, Polynesians, Native Americans and South Asian Indians. The variance (s²) was 0.0026 and the standard deviation (s) was a narrow 0.04773. The coefficient of variance was only 8.88%. The median was 0.4731 and the midrange was 0.4783. With 95% confidence it can be predicted that the mean population ROB mass for women exists between the values 0.4686 and 0.4818. Kurtosis was calculated by Σ(xi−μ)3/Nσ3, where—

μ=population mean

xi=sample

N=sample size

σ=population standard deviation

### Example 2

### Health Index Predicts Metabolic Syndrome

Metabolic syndrome is widely acknowledged by all medical organizations as a major indicator of disease. It is a cluster of conditions—increased blood pressure, high blood sugar, excess body fat around the waist, and abnormal cholesterol or triglyceride levels—that when occurring together, significantly increase your risk of heart disease, stroke and diabetes.

Having just one of these conditions is not metabolic syndrome. Having more than one of these increases your risk even more. You are considered positive for metabolic syndrome if you have positive for three or more conditions.

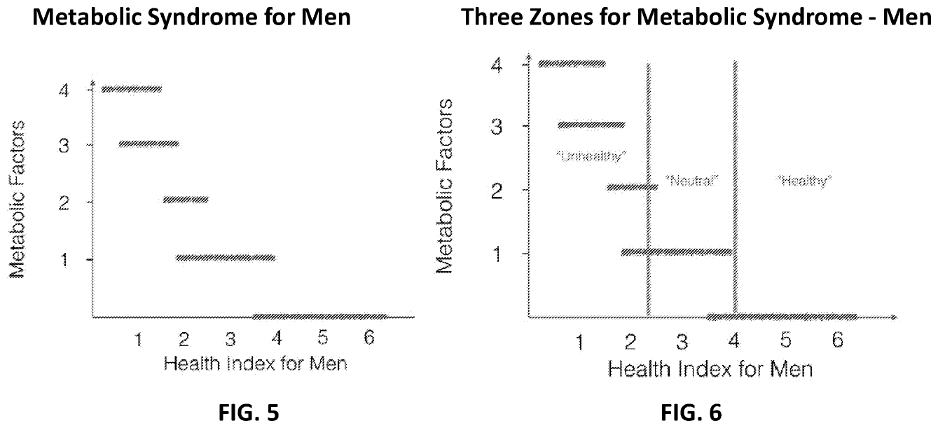

The correlation between Health Index for men can be seen in the following FIG. 5. There is an inverse correlation between Metabolic Syndrome and Health Index. As the Health Index of a man decreases the number of metabolic factors increase. Based on this data, three Health Index “zones” for men were created as depicted in FIG. 6. Above a Health Index of 4, a man is extremely healthy and is expected to have no metabolic conditions. Between a Health Index of 2.3 to 4, a man may have 0-2 metabolic conditions. If he is closer to 4 (example 3.8), he is expected to have no metabolic factors, but may have 1-2 metabolic conditions if his Health Index is 2.5. When a man's Health Index is trending below 2.3, he is in poor health.

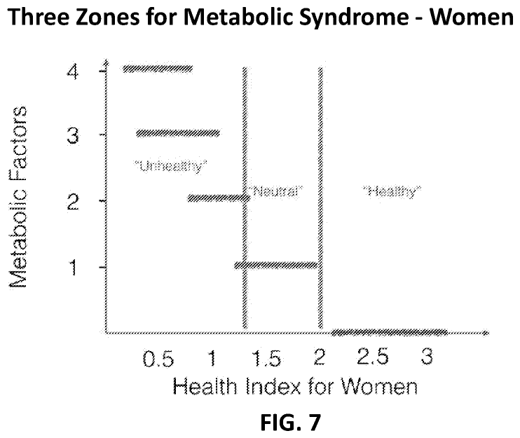

The corresponding correlation between Health Index and Metabolic Syndrome for women is shown FIG. 7. Above a Health Index of 2, a woman is extremely healthy and is expected to have no metabolic conditions. Between a Health Index of 1.4 to 2, a woman usually has 1-2 metabolic conditions. When a woman's Health Index is trending below 1.3, she is in poor health. Women's body has more fat and less muscle than a man's. Therefore their corresponding Health Index numbers are less. A woman's “neutral” zone is smaller than a man's. Women slip from a state of “health” to “ill health” more easily than men.

The curve for disease risk with decreasing Health Index, for both men and women, can be predicted with 99.92% accuracy (R2=0.9992) by the formula—

Disease Risk=−0.75x³+10.821x²−56.429x+106.2, where x=Health Index

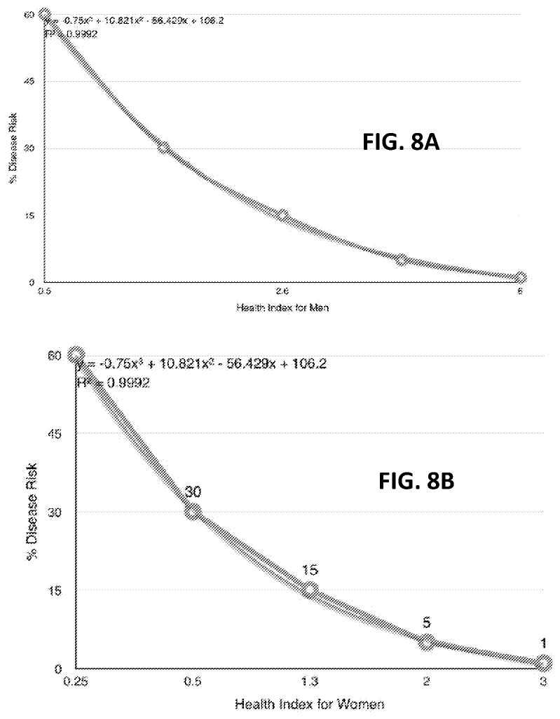

The curves for Disease Risk vs Health Index for Men and Women is shown in FIGS. 8A and 8B, respectfully.

### Example 3

### Health Index Provides a Significant Improvement over Body Mass Index (BMI)

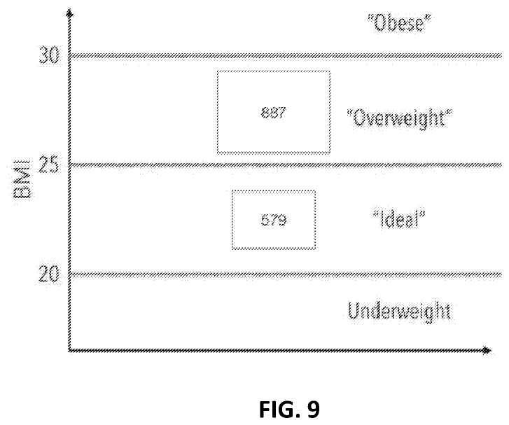

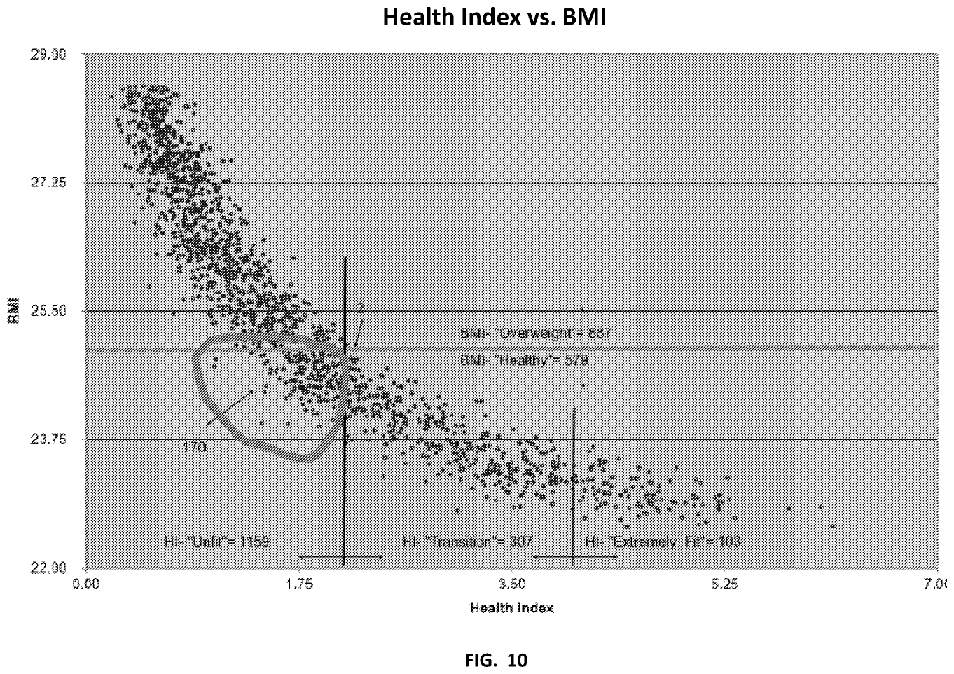

A group of 1466 men whose BMI were less than 30 were selected for this study. Of these men, 579 measured as “Ideal” BMI and 887 measured as “Overweight” . A breakdown of these men based on their BMI is illustrated in FIG. 9. None of the men who tested as “Obese” were selected because the health problems associated with BMI greater than 30 is not in dispute. Men who were less than 18.5 BMI (“Underweight”) were also not selected.

The same 1466 men were analyzed for their Health Index. Of this group, 103 men had a Health Index greater than 4 and were “Healthy”, and 307 had a Health Index between 2.4 and 4 and were in a transition zone between “Healthy” and “Unhealthy”. This transition zone information is important. If a person's Health Index is increasing from 2.4 to 4 then the person is becoming healthier. However, if their Health Index is decreasing from 4 to 2.4 then they are slipping into ill health.

FIG. 10 is a chart collating the health index and BMI of each subject in this study. Most critically, 170 men were flagged as “Unhealthy” because they had a Health Index score less than 2.4. However, they still had a BMI less than 25 which is considered “Ideal”. Health Index is a far more discerning measure of health than BMI. Health Index picks up the transition from Healthy to Unhealthy. BMI does not. BMI also classified 11.6% of the sample population as “Ideal” while the Health Index classified them as “Unhealthy”.

### Example 4

### Correlation of Health Index to Caloric Burn

The human anatomy is composed of numerous organs- brain, heart, arteries, veins, muscles, liver, gall bladder, kidney, skeleton, intestines, lymph nodes, lungs, spleen, bone marrow, stomach, pancreas, urinary system, reproductive organs, to name a few. The organs are made up of various tissues such as connective tissues, blood components, adipose or fatty tissue, connected bone tissue, cartilage and differing types of muscles. The tissue structure of individuals with high health index (HIX) differs significantly from individuals with low HIX. This is a significant component of health and it is going to be dealt in depth in this section.

Blood Utilization During Rest: An average 170 lb male has about 5 liters of blood in him. This blood is recirculated over and over again, as needed by the body. A person with low HIX (0.5) pumps about 4 liters of blood a minute during rest. A normal person, with a HIX of 3, pumps about 5 liters/min and a person with a HIX of 5 pumps about 6 liters/min of blood. This difference gets more interesting when one examines how it is used. A person with HIX of 0.5 uses 3.6 liters/min for the organs and only 0.4 liters/min for the skeletal system at rest. The quantity and quality of the skeletal muscle is low and therefore less blood is needed for the skeletal muscles and more is needed for the non-skeletal organs.

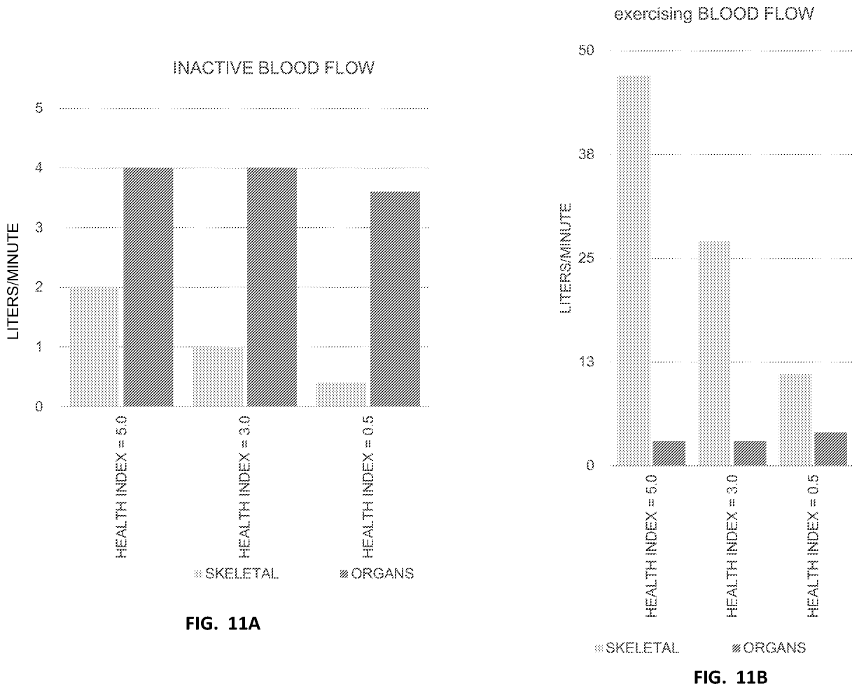

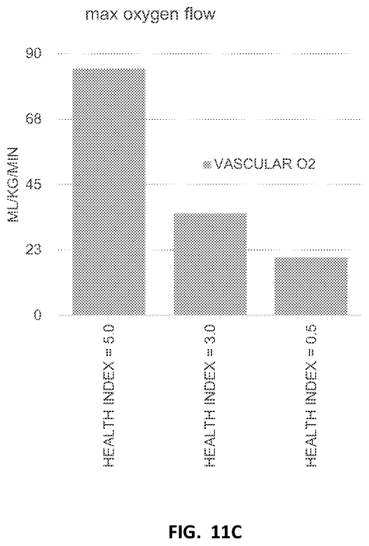

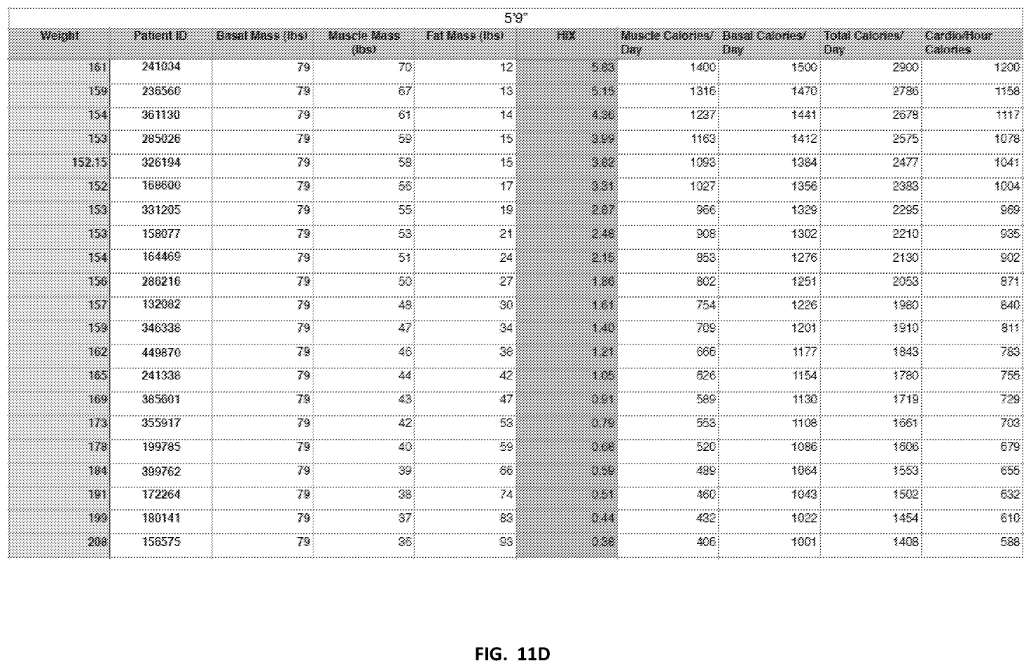

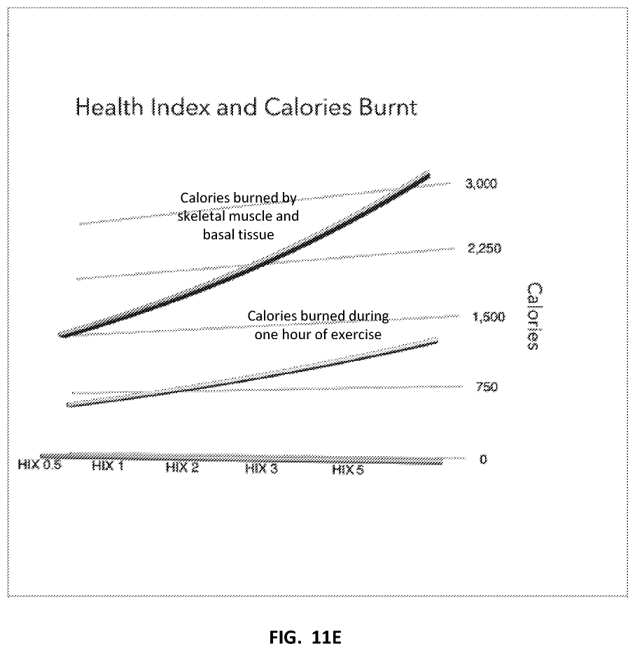

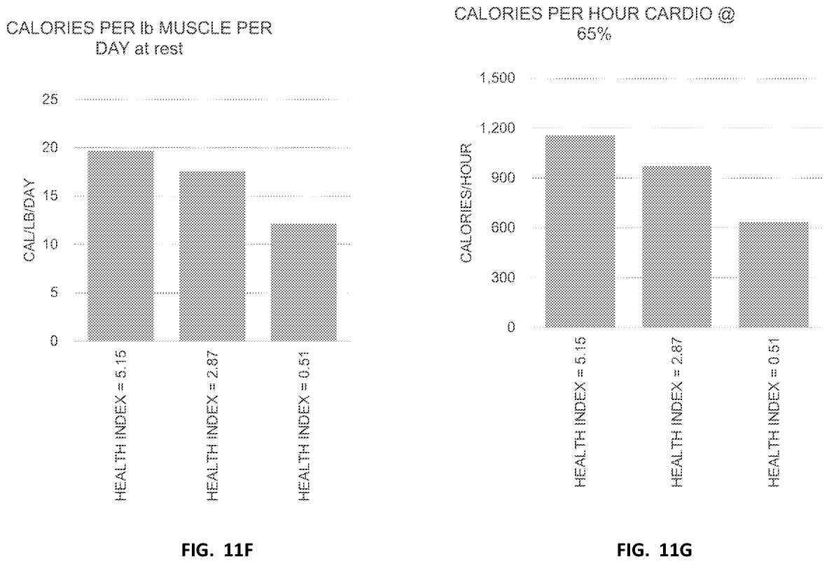

A person with HIX 5.0, at rest, pumps a bit more blood into his skeletal muscles (2 liters/min versus 0.4 liters/minute). But he still pumps more blood into his organs and there is not much difference between the overall profile of a person in peak health and an unhealthy person. FIG. 11A provides a graphical comparison of blood utilization during rest by individual males of the same height, but different health indices. This changes a lot when you compare a person with low HIX to a person with high HIX during maximal exertion.

Blood Utilization During Exercise or Maximal Exertion: A person with HIX of 0.5 pumps about 15 liters/min of blood when exerting. He pumps about 4 liters/min of blood through his organs (same as when resting) but his blood flow to his skeletal muscles increases from 0.4 liters/min to 11 liters/min, when he huffs and puffs and exerts.

A person with a HIX of 3 pumps about 3 liters/min through his organs and a significantly higher 27 liters/min of blood to his skeletal muscles when exerting. Here is where a person in peak health really differentiates himself. A high performance athlete, with a HIX of 5 or more, pumps a paltry 3 liters/min through his organs but a whopping 47 liters/min of blood through his muscles when exerting. This is why he is able to perform much better than a weak individual—with much less effort. FIG. 11B provides a graphical comparison of blood utilization during exercise by male individuals of the same height, but different health indices.

Blood performs numerous critical functions in the body. When the heart pumps a phenomenal amount of blood thorough the tissues, it is also pumping more oxygen. There is increased supply of nutrients such as glucose, amino acids, plasma proteins and blood lipids. There is a larger amount of waste produced by the muscles and removed by the blood, such as, carbon dioxide, urea and lactic acid. There is far greater immunological function by the body because foreign materials get detected sooner and the antibody response from the white blood cells is faster.

If there is any damage to the tissue, recognition and signaling the damage happens faster. Transport of hormones happens faster. The coagulation of leaking blood and the healing of the tissue happens faster too. Regulation of body temperature and pH happens quickly and more reliably. Finally, all the “hydraulic” elements of blood flow happen much faster and more reliably in a person who is pumping 47 liters/minute through his cardiovascular tissue than a person who is pumping 11 liters/min.

Oxygen Utilization During Exercise and Maximal Exertion: As discussed in the previous section, an increased blood flow means that increased oxygen flows thorough the tissues. Increased vascular development and oxygen utilization is a key indicator of health for all the reasons elaborated above.

A person with a low HIX of 0.5 usually has a weak cardiovascular system that carries only 20 ml/kg/min of oxygen. An average male, with a HIX of 3.0, pumps about 35 mil/kg/min of oxygen. A person with HIX greater than 5.0, in peak health, pumps about 85 ml/kg/min of oxygen. FIG. 11C provides a graphical comparison of oxygen flow in male individuals of the same height, but different health indices.

Calories Consumed by Various Health Indices: Based on the foregoing, it is clear that the quality of neuromuscular, vascular, skeletal and cardiac tissues for humans with high, medium and low Health Indices differs significantly. The table shown in FIG. 11D, contains data for individuals are males, all of the same height—5′9″. Column 1 is their weight. Column 2 is a “double blind” identification given for the individual. Column 3 is the basal mass (lbs) assumed for each individual. It is the same, since they are the same height. Column 4 is their Muscle Mass (lbs). Column 5 is their Fat Mass (lbs).

The individual with the ID 160316 has a HIX of 0.51. At rest, this individual's skeletal muscle burns 460 calories/day, or 12.1 calories/lb/day. The basal tissue (rest of the organs including the involuntary muscles) burns 1,043 calories/day, or 13.2 calories/lb/day (see FIG. 11F). Fat takes no calories to maintain. There is no vascular tissue flowing blood through it.

It is highly unlikely that this individual will not move during the day. So, their calories expended for 1 hour of cardio on a treadmill was measured at 65% of maximal heart rate. This was measured to be 632 calories, or 16.63 calories/lb skeletal muscle/hour (FIG. 11G).

The individual with the ID 266814 has a HIX of 2.87. At rest, this individual's skeletal muscles burns 966 calories/day, or 17.56 calories/lb/day. The basal tissue (rest of the organs including the involuntary muscles) burns 1,329 calories/day, or 16.82 calories/lb/day (FIG. 11F). His calories expended for 1 hour of cardio, on a treadmill at 65% of maximal heart rate, was 969 calories, or 17.62 calories/lb skeletal muscle/hour (FIG. 11G).

The individual with the ID 221038 has a HIX of 5.15. At rest, this individual's skeletal muscles burns 1,316 calories/day, or 19.64 calories/lb/day. The basal tissue (rest of the organs including the involuntary muscles) burns 1,470 calories/day, or 18.60 calories/lb/day (FIG. 11F). His calories expended for 1 hour of cardio, on a treadmill at 65% of maximal heart rate, was 1,158 calories, or 17.82 calories/lb skeletal muscle/hour (FIG. 11G).

In all these cases, the individuals with higher HIX had more skeletal muscles. Their skeletal muscles burnt more calories/hour, consumed more oxygen and had far more blood pumped into it during activity.

### Example 5

### Fabrication of Health Index Sensor with Pressure Transducer Component

Materials: Gold (III) chloride trihydrate (HAuCl4 3H2O, Z99.9%) and Triisopropylsilane (99%) were purchased from Sigma Aldrich. Research grade ethanol, hexane, and chloroform were purchased from Merck KGaA. All glassware used was cleaned in a bath of freshly prepared aqua regia and rinsed thoroughly in water before use. Graphene paper membrane material (10 microns, Tensile modulus 20 GPa) was purchased from Graphene Supermarket.

PDMS substrates were made by the mixing of the prepolymer gel (Sylgard 184 Silicone Elastomer Base) and the cross linker (Sylgard 184 Silicone Elastomer Curing Agent) at the weight ratio of 10:1. The mixture was poured on a 600 flat-plate petri dish using 0.5 mm-height shims as spacers and cured at 65° C for 2 h in an oven. After curing, the PDMS sheet with a thickness of 500 mm was cut into 3027 mm² strips for further treatment. The stainless shadow masks were purchased from MasterCut Techniques, Australia. Silver paste was from Dupont (Dupont 4929 N, DuPont Corporation, Wilmington, Del., USA). Aqueous CNT solutions (1 mg ml 1) with sodium dodecylbenzenesulphonate (SDBS) (1:10 in quality ratio) as a surfactant were prepared according to the Hu et al. “Highly Conductive Paper for Energy-storage Devices. Proc. Natl Acad. Sci. 106: 21490-21494 (2009), which is hereby incorporated by reference in its entirety. Gold nanorods were synthesized similar to the procedure used by Tang et al., “Lightweight, Flexible, Nanorod Electrode with High Electrocatalytic Activity,” Electrochem. Commun. 27:120-123 (2012), which is hereby incorporated by reference in its entirety.

Sensor fabrication was carried out essentially as described by Gong et al., “A Wearable and Highly Sensitive Pressure Sensor with Ultrathin Gold Wires,” Nature Comm. 5:3132 (2014), which is hereby incorporated by reference in its entirety, with modification. A schematic of the fabrication is depicted in FIG. 13. Briefly, 3 cm×10 μ graphene paper was dipped into a chloroform solution of the AuNWs. After evaporating the chloroform, the color of graphene paper changed from black to dark red. Then the dip-coating and drying process were repeated for about 10 cycles until the electrical resistance of paper sheets reached to ˜2.5 M Ω sq1.

This is where the various sensor fabrication happens. If the cycles and the thickness of the coat is increased, the sensor is less sensitive and can be tailored to sense macro level pressure, such as human weight. The thickness of the graphene layer, the coating thickness, the PDMS layer all affect sensor characteristics. Thus a large variety of sensors having different sensing capabilities can be made based on the AuNW coating thickness.

The Ti/Au interdigitated electrodes (thickness at 3 nm to 30 nm) were deposited onto PDMS substrates (3027 mm²) using a designed shadow mask by an electric beam evaporator (Intivac Nanochrome II, 10 kV). The spacing between the adjacent electrodes was typically 0.1 mm, with the width of interdigitated electrodes at 0.5 mm. Two 10×10 mm² contact pads were deposited at the two ends of the interdigitated electrodes to establish external contacts. Then, the bottom layer electrode coated PDMS and upper layer blank PDMS supports were treated by thin oxygen plasma (Harrick Plasma Cleaner PDC-001) and permanently sealed outside the AuNWs coated tissue paper to ensure conformal contact of tissue paper and interdigitated electrodes.

Large-area Fabrication and patterning was essentially carried out as described by Gong et al., “A Wearable and Highly Sensitive Pressure Sensor with Ultrathin Gold Wires,” Nature Comm. 5:3132 (2014), which is hereby incorporated by reference in its entirety. Briefly, a 5×5 cm² graphene paper was first dipped into the AuNWs stock solution and dried for 10 cycles, leading to a uniform dark red graphene paper. A patterned Ti/Au interdigitated electrode arrays on PDMS substrates (65×65 mm²) was fabricated using shadow mask lithography mentioned earlier. The spacing and width of electrodes were kept at 100 and 200 mm, with each interdigitated electrode pixel at 5×5 mm². Finally, each AuNWs impregnated graphene paper piece was addressed to the specific electrode pixels and sandwiched between the plasma treated PDMS sheets, leading to large-area, patterned pressure sensors.

### Example 6

### Fabrication Health Index Sensor with Impedance Component

Materials: TL 072 Op Amps x2; INA 182 Instrumentation Amplifier; AD5933 Chip; Resistors (100,000 ohms, 1000 ohms); Wires; Computer; Arduino with Micro USB cable; Breadboard; 4 Leads with Pads.

The circuit itself uses three op amps, an instrumentation amplifier and an AD5933 chip that interfaces with the software code to output a real and imaginary value that is converted to an impedance, which is then combined with height and weight information to give a final fat composition. It is very difficult to measure body fat even with today's technologies due to differing levels of bone and muscle mass between individuals. Some methods that exist now along with bioelectrical impedance analysis are underwater weighing, whole-body air displacement plethysmography, near-infrared interactance, body average density measurement, BMI, and other anthropometric methods. The largest obstacle in this process was ensuring that the current that ran through the body was comparable and safe with human tissue.

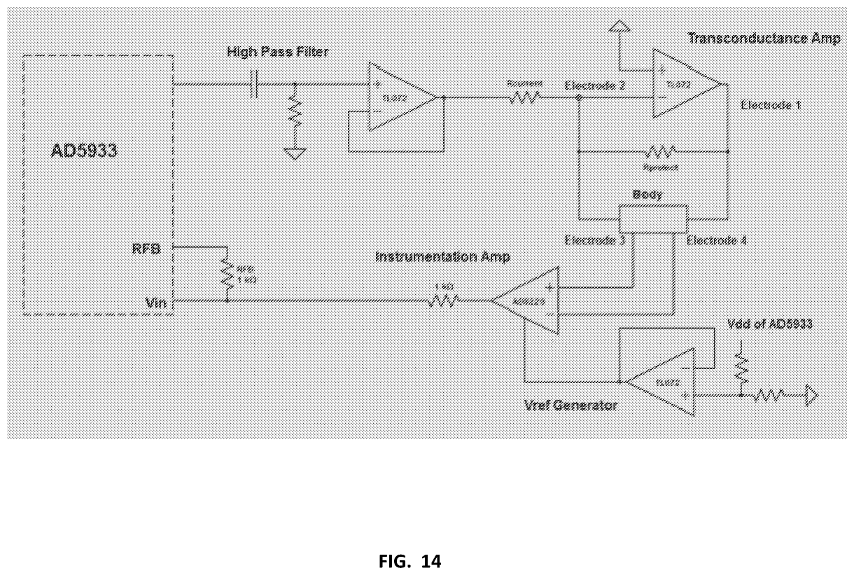

Each part of the circuit that is external to the AD5933 chip was tested multiple times to ensure that a current was not being produced above 10 microAmps which was determined to be a reasonable voltage. The high pass filter and transconductance amplifier was built first using a function generator to provide the voltage instead of the AD5933. The high pass filter with buffer composed a voltage follower using a 10\hat{ }5 pF capacitor with a 1000 ohm resistor that was connected to the positive terminal. The negative terminal ran to the output. All descriptions of the circuit can be visually conceptualized using the circuit diagram of FIG. 14.

Immediately following the filter is a voltage to current converter, as a current is necessary to run through the body. A 100,000 Ohm resistor was positioned in place of the body. The value of Rcurrent that was necessary to turn the supplied voltage into a 10 microAmp current was calculated to be approximately 200,000 Ohms which feeds into the negative terminal of the second op amp. The TL072 has two op amps integrated into it which is why there are only two op amps visible in the circuit (FIG. 14) despite the fact that three functional op amps were used. The negative terminal is also connected to the body using a lead in addition to the Rprotective resistor. The Rprotective is set to be x15 the resistance of the body resistance. A 1,500,000 ohm resistor was used based on the assumption of a 100,000 ohm resistance of the body which was chosen from research on typical body impedances in this capacity. A frequency sweep from 1000 Hz to 10,000 Hz using a sine wave with amplitude of 2 V peak to peak with an offset to keep it positive was used. All op amps were set with +/−10 V rails.

The instrumentation amplifier that followed the voltage to current converter required a reference voltage that was created with another TL072 op amp. This reference voltage needs to be half of the current inputted into the circuit. In this case, 5 V was being put into the circuit forcing Vref to be 2.5 V. A simple nodal analysis was used to find that in order to create 2.5 reference voltage the two resistors feeding into the op amp had to be equal. This V ref is fed into the instrumentation amplifier which adds this reference with the input voltage coming in from the body. The output of the instrumentation amplifier is followed by a 1000 ohm resistor which feeds into Pin 5 of our AD5933. This node also has an external feedback resistor (RFB) of 1000 ohms which is attached to the RFB Pin 4.

Once all of the circuit had been built and tested the AD5933 chip was added, and the circuit was powered with the Arduino. Pin 6 of this chip is Vout, and produces a 5V voltage which is used to power the circuit. Pins 4 and 5 were where the Rfeedback and Resistor of the instrumentation amplifier fed in, respectively. Pins 9, 10 and 11 are attached to the 5V source of the Arduino, 12, 13 and 14 are attached to ground, and pins 15 and 16 are attached to A4 and A5 of the Arduino, respectively. The circuit was then tested with the 100k resistor in place of the body to make sure an impedance was measured and the current was safe to run through the body.

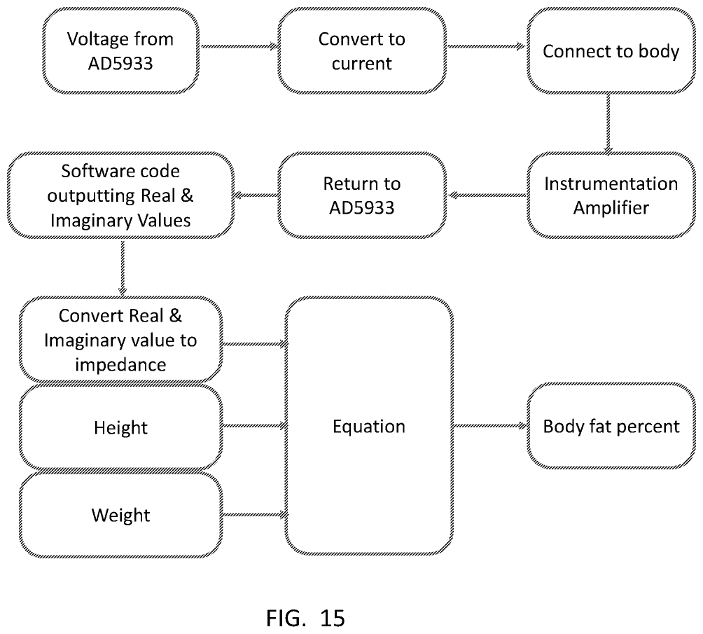

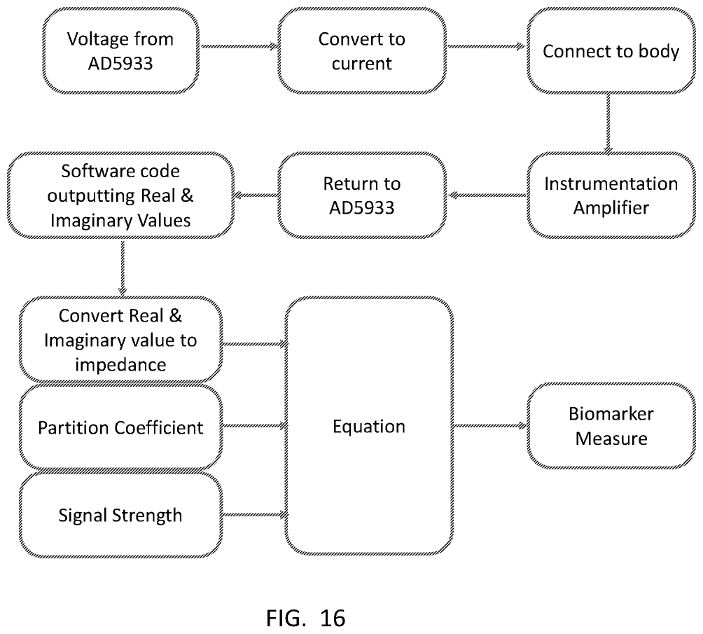

Finally, the leads were attached. The body has 4 leads connected to it, two of which are for current and two for voltage. One current and one voltage electrode was placed on the hand, and the remaining two were placed on the foot. The locations can be seen in the attached picture; leads 1 and 2 are current electrodes while 3 and 4 are the voltage electrodes. FIG. 15 is a flow diagram showing how body fat percentage is determined using the impedance circuit.

### Example 7

### Perimenopause Detection Based on Sweat Analysis

Perimenopause includes the period shortly before menopause and the first year after menopause, signalling the onset of the biological, hormonal and clinical symptoms characteristic of menopause. During perimenopause, there is frequent amenorrhea and or increased menstrual irregularities resulting from fluctuations in levels of hypothalamic, pituitary and ovarian hormones. It usually begins in a woman's late 40s, and continues until the cessation of ovulation and menses. It can last from only a few months to an average of 4-5 years. It terminates at the absence of menstrual flow for more than 12 months (menopause).

Detection of sweat potassium levels in eighty three (83) female volunteers comprising of Pre Menstrual (aged: 22.5±0.8 yrs, n=21), Peri Menstrual (aged: 46.5±1.1 yrs, n=43), and Post Menstrual (aged: 52.2±0.9 yrs, n=19) was carried using the sensor as essentially described in Example 5. Sweat was measured after a 15 min walk on a calibrated treadmill at a speed of 4.2 km/h at 27° C. and a relative humidity of 85-95%, followed by measurement of sweat volume (SV) and [K+] (see Amabebe et al., “Sweat Potassium Decreases with Increased Sweating in Perimenopausal Women,” British Journal of Medicine & Medical Research 14(2):1-10 (2016) (“Amabebe”), which is hereby incorporated by reference in its entirety). Sweat rate (SR) was determined by dividing the volume of sweat produced by the duration of exercise.

Results using the sensor described herein produced similar results as those described in Amabebe et al., “Sweat Potassium Decreases with Increased Sweating in Perimenopausal Women,” British Journal of Medicine & Medical Research 14(2):1-10 (2016), which is hereby incorporated by reference in its entirety). The PeriM women demonstrated higher SR (ml/min) (P=0.01) and SV (ml)

(P=0.0006) compared to women in the other groups: SR (PeriM=0.12±0.01; PreM=0.07±0.02; PostM=0.06±0.01), and SV (PeriM=1.7±0.2; PostM=0.9±0.1). However, they had lower sweat [K+] (mmol/l) (P=0.04), compared to their PostM counterparts (PeriM=19.98±1.5; PostM=24.90±1.8). Furthermore, sweat [K+] was inversely associated with SR (r=−0.4, P=0.02).

Although excessive sweating can lead to depletion of the body's potassium concentration, the sweat potassium concentration decreases with increased sweating in perimenopausal women. This could be an adaptive mechanism inhibiting excessive potassium loss.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A system for monitoring a subject's health index, said system comprising:
one or more wearable sensor units, each sensor unit comprising a pressure transducer component and an electrical impedance measuring component;
a network interface unit coupled to said one or more sensor units for transmitting data from the one or more sensor units;
a health index computing device coupled to the one or more sensors units for receiving data from the one or more sensor units, said health index computing device comprising a memory coupled to a processor which is configured to be capable of executing programmed instructions comprising and stored in the memory to:
determine one or more physiological parameters, based on said received data from the one or more sensor units, wherein said one or more physiological parameters include at least the subject's weight and the subject's body fat composition, and
measure the health index of the subject based on the determined weight and body fat composition of the subject, and the subject's rest of body mass.

2. The system of claim 1 further comprising:
a data storage unit coupled to said health analyzing computing device that is configured to receive and store information generated by said health analyzing computing device.

3. The system of claim 1, wherein the health index computing device further comprises:
a user interface, wherein the processor is further configured to be capable of executing additional programmed instructions comprising and stored in the memory to:
display said measured health index on said user interface.

4. The system of claim 1, wherein the processor of the health index computing device is further configured to be capable of executing additional programmed instructions comprising and stored in the memory to:
diagnose metabolic syndrome in the subject based on said measured health index.

5. The system of claim 1, wherein the health index computing device further comprises:
a microcontroller unit coupled to said one or more sensors.

6. The system of claim 1, wherein the health index computing device comprises a personal computing device.

7. The system of claim 6, wherein the personal computing device is a mobile telephone.

8. The system of claim 1, wherein the pressure transducer component of the one or more sensor units comprises ultra-thin gold nanowires (AuNW).

9. The system of claim 1, wherein the electrical impedance measuring component of the one or more sensor units consists of a single electrode configured to measure current flow.

10. The system of claim 1, wherein the one or more sensor units are configured to be placed in contact with at least a portion of the ventral surface of the subject's foot.

11. The system of claim 1, wherein the one or more sensor units are configured to be affixed to an insert of a shoe.

12. The system of claim 1, wherein the pressure transducer component of the one or more sensor units detects bending and torsional forces, and said one or more physiological parameters determined by said health index computing device includes the subject's gait.

13. The system of claim 1, wherein said one or more physiological parameters determined by said health index computing device includes blood pressure.

14. A method of measuring the health index of a subject, said method comprising:
receiving, by a health index computing device, data from one or more sensors worn by said subject, said one or more sensors comprising a pressure transducer unit and an electrical impedance measuring unit;
determining, by the health index computing device, one or more physiological parameters related to the subject's health index based on the received data, wherein said physiological parameters include at least the subject's weight and body fat composition; and
measuring, by the health index computing device, the health index of the subject based on the determined weight and body fat of the subject, and the subject's rest of body mass.

15. The method of claim 14 further comprising:
displaying, by the health index computing device, the measured health index on a user interface of the health index computing device.

16. The method of claim 14 further comprising:
transferring, by the health index computing device, the measured health index to a third party server device.

17. The method of claim 14, wherein said measuring comprises:
determining, by the health index computing device, the muscle mass of the subject by subtracting the subject's body fat and rest of body mass from the subject's body weight; and
calculating, by the health index computing device, the health index of the subject by dividing the determined muscle mass of the subject by the subject's body fat.

18. The method of claim 14, wherein the one or more physiological parameters further include blood pressure and/or gait of the subject.

19. A non-transitory computer readable medium having stored thereon instructions for calculating a subject's health index comprising executable code which when executed by a processor, causes the processor to perform steps comprising:
receiving data from one or more sensor units worn by said subject, said one or more sensor units each comprising a pressure transducer unit and an electrical impedance measuring unit;
determining one or more physiological parameters related to the subject's health index based on the received data, wherein said physiological parameters include at least the subject's weight and body fat composition; and
measuring the health index of the subject based on the determined weight and body fat of the subject, and the subject's rest of body mass.

20. The medium of claim 19 further having stored thereon at least one additional instruction comprising executable code which when executed by the processor, causes the process to perform additional steps comprising:
displaying the measured health index on a user interface of the health index computing device.

21. The medium of claim 19 further having stored thereon at least one additional instruction comprising executable code which when executed by the processor, causes the process to perform additional steps comprising:
transferring the measured health index to a server device.

22. The medium of claim 19 further having stored thereon at least one additional instruction comprising executable code which when executed by the processor, causes the process to perform additional steps comprising:
diagnose metabolic syndrome in the subject based on said measured health index

23. A health index computing device comprising:
a processor;
a memory coupled to the processor which is configured to be capable of executing programmed instructions comprising and stored in the memory to:
receive data from one or more sensor units worn by a subject, said one or more sensor units each comprising a pressure transducer unit and an electrical impedance measuring unit;
determine one or more physiological parameters related to the subject's health index based on the received data, wherein said physiological parameters include at least the subject's weight and body fat composition; and
measure the health index of the subject based on the determined weight and body fat of the subject, and the subject's rest of body mass.

24. The device of claim 23, wherein the memory coupled to the processor which is further configured to be capable of executing programmed instructions comprising and stored in the memory to:
display the measured health index on a user interface of the health index computing device.

25. The device of claim 23, wherein the memory coupled to the processor which is further configured to be capable of executing programmed instructions comprising and stored in the memory to:
transfer the measured health index to a third party server device.

26. The device of claim 23, wherein the memory coupled to the processor which is further configured to be capable of executing programmed instructions comprising and stored in the memory to:
diagnose metabolic syndrome in the subject based on said measured health index.
